(19) United States
(12) Patent Application Publication
(10) Pub. No.: US 2011/0046532 A1
(43) Pub. Date: Feb. 24, 2011

(54) COMPOSITION INCLUDING A SILICONE-BASED POLYMER AND A METHOD OF TREATING SKIN DISORDERS USING THE COMPOSITION

(76) Inventors: Sanjeev Kaila, Sarnia (CA); John Milne, Sarnia (CA)

Correspondence Address: HARNESS, DICKEY & PIERCE, P.L.C., P.O. BOX 828, BLOOMFIELD HILLS, MI, 48303, US

(21) Appl. No.: 12/223,202

(22) PCT Filed: Jan. 29, 2007
(86) PCT No.: PCT/CA2007/000110

§ 371 Date: Oct. 22, 2010

Related U.S. Application Data
(60) Provisional application No. 60/763,492, filed on Jan. 30, 2006.
(60) Provisional application No. 60/774,894, filed on Feb. 17, 2006.

(51) Int. Cl. A61F 13/00; A61K 31/80; A61P 17/02; A61P 17/10; A61P 17/06

(52) U.S. Cl. 602/48; 424/78.06

(57) ABSTRACT

A composition is formed by the step of feeding a molecule that has an active oxygen-forming species into a silicone based polymer. The silicone-based polymer is substantially free of pendant groups that are reactive with active oxygen species. Silicone-based polymers have proven scar-healing properties. As such, the composition is useful for treating skin disorders, especially scars, to reduce the appearance of the scars. Furthermore, bacteria and infections may contribute to the formation of scars. The active oxygen-forming species form active oxygen species, which oxidize pathogens including bacteria, viruses, and fungus to sterilize an area in contact with the composition. As a result, the appearance of scars treated with the composition may be even further reduced due to the presence of the molecule having the active oxygen-forming species.

### CROSS-REFERENCE TO RELATED APPLICATIONS

This application claims the benefit of U.S. Provisional Patent Application No. 60/763,492 filed Jan. 30, 2006 and U.S. Provisional Patent Application No. 60/774,894 filed Feb. 17, 2006, the content of both of which is incorporated by reference herein.

### FIELD OF THE INVENTION

The subject invention relates to a composition and a method for treating skin disorders using the composition. More specifically, the composition includes an ozonated silicone-based polymer that may be used for treating the skin disorders.

### BACKGROUND OF THE INVENTION

Silicone-based polymers are well known in the art and are used in a wide range of applications. One example of a common use for silicone-based polymers is for treating both keloid and hypertrophic scars resulting from burns, surgery, or minor skin injuries. The use of the silicone-based polymers has been proven to reduce the appearance of the scars when used for long periods of time. Although the specific mechanism through which silicone-based polymers function to reduce the appearance of scars is not presently known, it has been hypothesized that the silicone mimics a natural barrier function of skin to promote the healing process. Other theories of why silicone-based polymers are so effective in reducing the appearance of scars revolve around increased hydration, oxygen tension, and the presence of silicone around the scar. When the silicone-based polymer is in the form of a solid sheet, it is possible that the sheet produces a static electric field that might have an effect on reducing the appearance of the scar. Numerous commercial products formed from silicone-based polymers and specifically targeting treatment of scars are presently available. Specific examples of such commercial products include Neosporin® Scar Solutions™ sheets, Mepiform® scar dressings, and Dermatix™ silicone gel.

Although the silicone-based polymers are effective in reducing the appearance of scars, the silicone-based polymers are typically applied to the scars for long periods of time. More specifically, the silicone-based polymers are typically worn on the scars for about 18 hours per day, every day, for months at a time to achieve noticeable results. Significant discomfort may result from the long periods during which the silicone-based polymers must be worn on the scar. As such, it would be advantageous to provide a composition that reduces the appearance of scars faster and more effectively than the silicone-based polymers alone.

Separate from the treatment of scars, other classes of compositions are widely used for treating open wounds and, in particular, preventing infections of the open wounds. To prevent the infections, the compositions typically include an active ingredient that kills bacteria. For example, Neosporin®, which is one commercially available composition for treating wounds that is widely used, includes various antibiotics such as bacitracin, neomycin, and polymixen. The antibiotics are mixed into a carrier composition that includes natural oils, such as cocoa butter and olive oil, as well as synthetic components, such as white petrolatum. The carrier composition does not possess the scar-healing properties of the silicone-based polymers, and excessive use of antibiotics may give rise to antibiotic resistant strains of bacteria, which is undesirable.

Ozone has also been used, both as a gas and in compositions of natural oils, as the active ingredient to kill pathogens and prevent infection in the open wounds. Ozone is a strong oxidizing agent and has excellent anti-pathogenic properties. When mixed with natural oils, such as macadamia nut oil or olive oil, the ozone reacts with organic components in the natural oil to form ozonides and ozone esters, which are oxidizing agents. The motivation behind using the natural oils is to provide the organic components that are reactive with the ozone in order to bind the ozone within the composition. However, the natural oils themselves do not provide significant scar-healing properties.

Thus, there remains an opportunity to provide a composition that is useful for treating skin disorders, especially scars, that both accelerates healing of the skin disorders so as to decrease the amount of time for which the composition must be worn on the skin disorders and that also reduces the appearance of the scars more effectively than known compositions.

### SUMMARY OF THE INVENTION

The subject invention provides a composition formed by the step of feeding a molecule that has an active oxygen-forming species into a silicone-based polymer. The silicone-based polymer is substantially free of pendant groups that are reactive with active oxygen species. The subject invention also provides a method of treating a skin blemish comprising the step of applying the composition including the silicone-based polymer and the molecule having the active oxygen-forming species onto the skin disorder.

The composition of the subject invention includes the silicone-based polymer, which has proven scar-healing properties. As such, the composition is useful for treating skin disorders, especially scars, to reduce the appearance of the scars. Furthermore, it is hypothesized that bacteria and infections contribute to the inflammatory process of healing and the formation of scars. The active oxygen-forming species form active oxygen species, which oxidize pathogens including bacteria, viruses, and fungus to sterilize an area in contact with the composition. As a result, it is hypothesized that the appearance of scars treated with the composition of the subject invention may be even further reduced due to the presence of the molecule having the active oxygen-forming species. Furthermore, by sterilizing the area in contact with the composition, healing of the skin disorders may be accelerated and the amount of time for which the composition must be worn on the skin may be decreased, as compared to usage times for silicone-based polymers alone.

### DETAILED DESCRIPTION

A composition is provided herein that is useful for applying to skin or mucosal surfaces to reduce the appearance of scars. Skin disorders that may be treated with the composition of the subject invention include, but are not limited to, scars, acne, acne scarring, athletes foot, psoriasis, eczema, and other skin conditions. Ozonated silicone can be used in acne medicine, suntan lotion, toothpaste, soap, shampoo, mouthwash, contraception, prevention of STD (sexually transmitted disease) transmission. The composition includes a silicone-based polymer and is formed through the step of feeding a molecule having an active oxygen-forming species into the silicone-based polymer. The silicone-based polymer including the molecule having the active oxygen-forming species combines proven advantages provided by silicone-based polymers for reducing the appearance of scars with advantages associated with active oxygen-forming species, i.e., oxidizing agents, for sterilizing and killing pathogens at a location of the skin disorder. By sterilizing and killing pathogens for the purpose of reducing the appearance of scars, as opposed to merely preventing infection, it is hypothesized that the appearance of the scars may be even further reduced due to the presence of the active oxygen-forming species.

Silicone-based polymers that are suitable for purposes of the subject invention typically have the following structure:

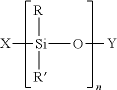

wherein R, R′, X, and Y each comprise a group that is non-reactive with active oxygen and n is at least 2. Such silicone-based polymers are termed polysiloxanes. One example of a polysiloxane that is suitable for the subject invention is poly(dimethyl siloxane), in which R and R′ are both methyl groups. Other suitable silicone-based polymers may include silanes or other silicone-based polymers that are known in the art.

The silicone-based polymer is substantially free of pendant groups that are reactive with active oxygen species. More specifically, pendant groups that are reactive with active oxygen species, such as vinyl groups, may result in unwanted crosslinking within the silicone-based polymer. Unwanted crosslinking may modify a viscosity and/or consistency of the silicone-based polymer while decreasing an amount of active oxygen species and active oxygen-forming species present in the silicone-based polymer after feeding the molecule having the active oxygen-forming species into the polymer. However, small amounts of pendant groups that are reactive with active oxygen species may be present in the silicone-based polymer, depending on the specific application. For example, pendant groups that are reactive with active oxygen species may be present in the silicone-based polymer in an amount of less than 0.15 parts by weight, preferably less than 0.05 parts by weight, based on the total weight of the silicone-based polymer.

Examples of specific silicone-based polymers that are particularly suitable for purposes of the subject invention include 100% poly(dimethyl siloxane) having a number average molecular weight of from 28,000 to 139,000, which is commercially available from Clearco Products Co., Inc., of Bensalem, Pa. Preferably, the silicone-based polymer is present in an amount of at least 95 parts by weight based on the total weight of the composition. However, it is to be appreciated that lower amounts of the silicone-based polymer may be present depending on the specific application.

The molecule having the active oxygen-forming species has an active oxygen content of at least 10%, preferably at least 20%, which correlates to high oxidative activity.

Such high active oxygen content makes the active oxygen-forming species an excellent anti-pathogenic agent.

The active oxygen content is defined as follows:

$$Active Oxygen Content = \frac{16 \times Number of Active Oxygen \text{-} Forming Species}{Number Average Molecular Weight of Molecule} \times 100$$

In one embodiment, the molecule having the active oxygen-forming species is ozone. The ozone may be produced from medical grade oxygen using an ozonator, such as those commonly used for dental applications. An active oxygen content of pure ozone, for example, is 33.33, since ozone has a number average molecular weight of about 48 and each ozone molecule has one active oxygen-forming species. In another embodiment, the active oxygen-forming species may be selected from the group of a hydroxy radical-forming species, a peroxy radical-forming species, an alkoxy radical-forming species, a hydroperoxide-forming species, and combinations thereof.

A specific amount of molecules having the active oxygen-forming species present in the composition is not easily measurable. More specifically, some of the molecules may decompose to produce the active oxygen species within the composition, some of the molecules may react with pendant groups on the silicone-based polymer that are reactive with active oxygen species, and some of the molecules may escape during formation of the composition or afterwards. In each of the above situations, there is a variable amount of molecules having the active oxygen-forming species, and thus, an amount of active oxygen species, present in the composition. For objective purposes, the amount of molecules having the active oxygen-forming species present in the composition is correlated to a rate at which the molecules are fed into a set amount of the silicone-based polymer over a set period of time. Specific rates are described in further detail below.

As set forth above, the composition may also include an additive, such as wax and/or another polymer, in order to adjust a viscosity of the composition. Additives suitable for purposes of the subject invention may include paraffin wax, petroleum jelly, alginate, hydrogenated soyabean oil, propylene glycol, cellulose, methyl cellulose, glycerine, Carrageenan, cellulose gum, and xanthan gum, guar gum and combinations thereof. Some of the additives may have a melting temperature above ambient temperature of about 25° C. The additive, when included in the composition, is typically present in an amount of from 1 to 99 parts by weight based on the total weight of the composition. When the additive is present, the amount of the silicone-based polymer present in the composition may be less than 95 parts by weight based on the total weight of the composition.

In one embodiment, the composition including the silicone-based polymer has a viscosity of less than or equal to about 2,000,000 centistokes at ambient temperature. Preferably, in this embodiment, the viscosity of the composition is less than 100,000 centistokes, more preferably from 1,000 to 100,000 centistokes at ambient temperature. To obtain the desired viscosity in the composition, silicone-based polymers having viscosities of less than or equal to about 2,000,000 centistokes are used. Silicone-based polymers having a viscosity closer to 2,000,000 centistokes have a consistency like that of honey or a thick gel. Silicone-based polymers having a viscosity closer to 1,000 centistokes have an oily consistency. Skin disorder dressings formed from the composition having the viscosity of less than 2,000,000 centistokes at ambient temperature are typically in the form of a gel or oil, and may be applied to the surface by spreading the composition onto the skin disorder. The skin disorder may then be covered with gauze or a bandage, or may be left uncovered.

In another embodiment, the composition including the silicone-based polymer resists flowing at ambient temperature of about 25° C. More specifically, the composition is solid at ambient temperature. To obtain such a composition, the silicone-based polymer may include the pendant groups that are reactive with the active oxygen species in the amount of less than 0.15 parts by weight based on the total weight of the silicone-based polymer. The silicone-based polymer including the pendant groups that are reactive with the active oxygen species may experience light crosslinking due to reaction with the active oxygen species, which may increase the viscosity of the composition, possibly to the point that the composition resists flowing at ambient temperature. Alternatively, as set forth above, the additive may be included along with the silicone-based polymer in such an amount that the resulting composition resists flowing at ambient temperature of about 25° C. Dressings that are formed from the composition that resists flowing at ambient temperature are typically in the form of a solid sheet that may be placed onto the skin or mucosal surface. In addition, a sheet of fabric may be adhered to the composition to provide for easy application of the composition onto the skin or mucosal surface.

The viscosity of the composition is dependent, in large part, on a molecular weight of the silicone-based polymer included in the composition. More specifically, poly(dimethyl siloxane) that has a viscosity of about 100,000 centistokes also has a number average molecular weight of about 139,000, and poly(dimethyl siloxane) that has a viscosity of about 1,000 centistokes also has a number average molecular weight of about 28,000. Of course, it is to be appreciated that the viscosity of the silicone-based polymers may be modified by possible crosslinking when the pendant groups that are reactive with the active oxygen species are present.

As mentioned above, the composition is formed by the step of feeding the molecule having the active oxygen-forming species into the silicone-based polymer. More specifically, the silicone-based polymer, optionally along with one or more of the additives, is placed into a vessel. A temperature of the silicone-based polymer is typically maintained at a temperature at least equal to a melting temperature of the silicone-based polymer during the step of feeding the molecule into the silicone-based polymer. For example, if a melting temperature of the silicone-based polymer is below ambient temperature of about 25° C., the step of feeding the molecule having the active oxygen-forming species into the silicone-based polymer may occur at about ambient temperature. Optionally, when the molecule is gaseous in form, the temperature of the silicone-based polymer may be decreased to just above the melting temperature of the silicone-based polymer to maximize the amount of the molecule present in the composition. This is due to the known principle that a saturation point of a gas in a liquid rises as a temperature of the liquid is decreased. On the other hand, if the melting temperature of the silicone-based polymer is above ambient temperature of about 25° C., a temperature of the silicone-based polymer within the vessel is raised to above the melting temperature of the silicone-based polymer. When additives, such as waxes or other polymers, are present with the silicone-based polymer in the vessel, the temperature may be maintained above the melting temperature of all components in the vessel.

The temperature of the silicone-based polymer within the vessel may be raised through any known method as known in the art, such as by applying heat to the vessel or inserting a heating element into the silicone-based polymer in the vessel. The molecule having the active oxygen-forming species is then fed into the silicone-based polymer, which is liquid in form.

The molecule having the active oxygen-forming species is typically fed into the silicone-based polymer at a rate of at least 0.00001 grams per gram of the silicone-based polymer per minute. In a preferred embodiment, the molecule having the active oxygen-forming species is fed into the silicone-based polymer at a rate of from about 0.01 to about 2, more preferably at a rate of about 1, grams per gram of the silicone-based polymer per minute. Preferably, the period of time for which the molecule is fed into the silicone-based polymer is at least 1 minute, more preferably from 50 to 100 hours.

Temperature is also a factor that contributes to the amount of the molecule having the active oxygen-forming species present in the composition when the silicone-based polymer is liquid in form. As addressed above, when the molecule having the active oxygen-forming species is gaseous in form, the temperature of the liquid silicone-based polymer may be decreased to increase the saturation point of the molecule in the silicone-based polymer, and that temperature may be maintained while feeding the molecule into the silicone-based polymer. Furthermore, a storage temperature of the composition including the silicone-based polymer and the molecule having the active oxygen-forming species is preferably kept as low as possible to minimize loss of the molecule having the active oxygen-forming species from the composition.

Feeding the molecule having the active oxygen-forming species into the silicone-based polymer results in the silicone-based polymer becoming infused with the molecule having the active oxygen-forming species and, possibly, active oxygen species formed from decomposition of the active oxygen-forming species. It is also possible that pendant groups that are reactive with active oxygen species may be present, as set forth above, in the silicone-based polymer. The pendant groups may react with the active oxygen-forming species.

The molecule having the active oxygen-forming species may be fed into the silicone-based polymer in a number of ways. For example, in one embodiment, the molecule having the active oxygen-forming species may be liquid in form, and the molecule having the active oxygen-forming species may be fed into the vessel, either before or after placing the silicone-based polymer into the vessel, and mixed with the silicone-based polymer in the vessel. In another embodiment, the molecule having the active oxygen-forming species may be gaseous in form. The vessel may have at least one port in a bottom thereof where the molecule having the active oxygen-forming species may be fed into the vessel and into the silicone-based polymer in the vessel. Alternatively, a tube may be placed into the silicone-based polymer in the vessel, with the molecule having the active oxygen-forming species fed through the tube and into the silicone-based polymer. When the molecule having the active oxygen-forming species is ozone, which is typically gaseous in form, the ozone gas may be produced using the ozonator and bubbled into the silicone-based polymer.

The following examples, as presented herein, are intended to illustrate and not limit the invention.

### Example 1

A composition in accordance with the subject invention is made by first placing 1000 grams of a silicone-based polymer into a glass beaker having a volume of about 1 liters. The silicone-based polymer is formed from 100 parts by weight poly(dimethyl siloxane) having a number average molecular weight of about 28,000 and a viscosity of about 1,000 centistokes. A melting temperature of the silicone-based polymer is about −47° C. As such, the silicone-based polymer is liquid in form at ambient temperature of about 25° C., and is maintained at about ambient temperature. A molecule having an active oxygen-forming species is then fed into the silicone-based polymer. More specifically, the molecule having the active oxygen-forming species is ozone, and is fed into the silicone-based polymer in the glass beaker as a gas at a rate of 0.1 grams per gram of the silicone-based polymer per minute for a period of about 72 hours. The ozone gas is generated with a dental ozonator from medical grade oxygen, and is fed into the silicone-based polymer by way of a tube extending from the dental ozonator into the silicone-based polymer in the glass beaker. Once the ozone gas is fed into the silicone-based polymer to form the composition, the composition is removed from the glass beaker and poured into a sealed container.

### Example 2

Another composition in accordance with the subject invention is made by first placing 1000 grams of a silicone-based polymer into a glass beaker having a volume of about 1 liters. The silicone-based polymer is formed from 100 parts by weight poly(dimethyl siloxane) having a number average molecular weight of about 139 000 and a viscosity of about 2000000 centistokes. Paraffin wax is added to the silicone-based polymer in the glass beaker in an amount of 200 grams. The paraffin wax is solid in form at ambient temperature of about 25° C. As such, heat is applied to the glass beaker using a hotplate until the paraffin wax becomes liquid in form. The paraffin wax and the silicone-based polymer are mixed together to form a homogenous mixture. Ozone is then fed into the mixture in the glass beaker as a gas at a rate of 0.1 grams per gram of the silicone-based polymer per minute for a period of about 72 hours. The ozone gas is generated with a dental ozonator from medical grade oxygen, and is fed into the silicone-based polymer by way of a tube extending from the dental ozonator into the silicone-based polymer in the glass beaker. Once the ozone gas is fed into the silicone-based polymer to form the composition, the composition is poured out of the glass beaker, formed into a sheet, and cooled to ambient temperature of about 25° C. to solidify the sheet.

The invention has been described in an illustrative manner, and it is to be understood that the terminology which has been used is intended to be in the nature of words of description rather than of limitation. Obviously, many modifications and variations of the present invention are possible in light of the above teachings, and the invention may be practiced otherwise than as specifically described.

1. A composition formed by the step of;
feeding a molecule having an active oxygen-forming species into a silicone-based polymer substantially free of pendant groups that are reactive with active oxygen species, said molecule having an active oxygen content of at least 10%.

2. A composition as set forth in claim 1 wherein said pendant groups that are reactive with active oxygen species are present in said silicone-based polymer in an amount of less than 0.15 parts by weight based on the total weight of said silicone-based polymer.

3. A composition as set forth in claim 1 wherein said molecule is ozone.

4. A composition as set forth in claim 3 wherein the step of feeding said molecule into said silicone-based polymer comprises bubbling ozone gas into said silicone-based polymer.

5. A composition as set forth in claim 1 further comprising the step of maintaining said silicone-based polymer at a temperature at least equal to a melting temperature of said silicone-based polymer during said step of feeding said molecule into said silicone-based polymer.

6. A composition as set forth in claim 5 wherein the step of feeding said molecule comprises feeding said molecule into said silicone-based polymer at a rate of at least 0.00001 grams per gram of said silicone-based polymer per minute for a period of at least 1 minute.

7. A composition as set forth in claim 1 wherein said silicone-based polymer has the structure: 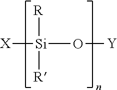
wherein R, R′, X, and Y each comprise a group that is non-reactive with active oxygen and n is at least 2.

8. A composition as set forth in claim 1 wherein said silicone-based polymer is present in an amount of at least 95 parts by weight based on the total weight of said composition.

9. A composition as set forth in claim 1 having a viscosity of less than or equal to about 2,000,000 centistokes at ambient temperature.

10. A skin disorder dressing formed from said composition as set forth in claim 9.

11. A composition as set forth in claim 1 that resists flowing at ambient temperature.

12. A skin disorder dressing formed from said composition as set forth in claim 11.

13. A skin disorder dressing as set forth in claim 12 further comprising a sheet of fabric adhered to said composition.

14. A method of treating a skin disorder comprising the step of;
applying a composition comprising a silicone-based polymer and a molecule having an active oxygen-forming species onto skin affected by said disorder.

15. A method as set forth in claim 14 wherein the molecule has an active oxygen content of at least 10%.

16. A method as set forth in claim 14 wherein the molecule is ozone.

17. A method as set forth in claim 14 wherein the silicone-based polymer is present in an amount of at least 95 parts by weight based on the total weight of the composition.

18. A method as set forth in claim 17 wherein the composition has a viscosity of less than or equal to about 2,000,000 centistokes.

19. A method as set forth in claim 17 wherein the composition resists flowing.

20. A method as set forth in claim 19 further comprising the step of adhering a sheet of fabric to the composition.

21. A method as set forth in claim 14, wherein said skin disorder is a scar, acne, acne scarring, athletes foot, psoriasis or eczema.

22. A composition for the treatment of skin disorders, said composition comprising: a molecule having an active oxygen-forming species, said molecule having an active oxygen content of at least 10%; and a silicone-based polymer substantially free of pendant groups that are reactive with said active oxygen species.

23. A composition as set forth in claim 22 wherein said pendant groups that are reactive with active oxygen species are present in said silicone-based polymer in an amount of less than 0.15 parts by weight based on the total weight of said silicone-based polymer.

24. A composition as set forth in claim 22 wherein said molecule is ozone.

25. A composition as set forth in claim 22 wherein said silicone-based polymer has the structure: 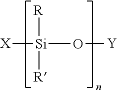
wherein R, R′, X, and Y each comprise a group that is non-reactive with active oxygen and n is at least 2.

26. A composition as set forth in claim 22 wherein said silicone-based polymer is present in an amount of at least 95 parts by weight based on the total weight of said composition.

27. A composition as set forth in claim 22 having a viscosity of less than or equal to about 2,000,000 centistokes at ambient temperature.

28. A composition as set forth in claim 22 that resists flowing at ambient temperature.
